(19) United States
(12) Patent Application Publication
(10) Pub. No.: US 2009/0203049 A1
(43) Pub. Date: Aug. 13, 2009

(54) BETA INTEGRIN GENE AND PROTEIN

(76) Inventors: Lee A. BULLA, Pilot Point, TX (US); Mehmet Candas, Dallas, TX (US)

Correspondence Address: MORRISON & FOERSTER LLP, 12531 HIGH BLUFF DRIVE, SUITE 100, SAN DIEGO, CA, 92130-2040, US

(21) Appl. No.: 12/356,508

(22) Filed: Jan. 20, 2009

Related U.S. Application Data
(63) Continuation of application No. 10/999,782, filed on Nov. 29, 2004.
(60) Provisional application No. 60/527,072, filed on Dec. 3, 2003.

(51) Int. Cl. G01N 33/53

(52) U.S. Cl. 435/7.21; 435/7.31

(57) ABSTRACT

Recombinant materials for the production of β integrin protein characteristic of Manduca sexta are useful for screening pesticides for this moth.

This application is a continuation of U.S. Ser. No. 10/999,782 filed 29 Nov. 2004, which claims priority under 35 U.S.C. § 119 from provisional application Ser. No. 60/527,072 filed 3 Dec. 2003. The entire contents of this document is incorporated herein by reference.

### TECHNICAL FIELD

The present invention relates to a previously unknown β integrin protein associated with insect midgut. This integrin protein is useful as a screening tool for pesticides.

### BACKGROUND ART

PCT publication WO 01/31011, published 3 May 2001, describes a “gene mining” system to permit identification of new genes based on phenotypic characteristics. This PCT publication is based on an application filed as PCT/US00/29445 filed 25 Oct. 2000, claiming benefit of U.S. application 60/161,527 set forth above. The contents of this application are incorporated herein by reference.

Briefly, this publication and the applications set forth above describe methods to retrieve nucleic acid sequences that encode a selected phenotypic characteristic in a selected species by designing primers based on nucleotide sequences that encode proteins that provide that characteristic in species that are known. The method comprises comparing a known protein sequence that effect a phenotype to a database of known protein sequences corresponding to the selected phenotypic characteristic and designing primers based on alignments of these sequences. The designed primers can then be used to amplify the appropriate sequences from any species for which the desired nucleotide sequence is unknown. This method was used successfully to obtain the nucleotide sequence and deduced amino acid sequence for the β integrin protein in the insect Manduca sexta.

### DISCLOSURE OF THE INVENTION

Manduca sexta (M. sexta) is a moth that infests tobacco; the caterpillars are known as tobacco hornworms and quickly defoliate tobacco plants. The moth is also known as Carolina sphinx. Since these organisms cause extensive crop damage to tobacco, a suitable pest control agent would be desirable. The present invention provides a β integrin target for identification of such agents.

Thus, in one aspect, the invention is directed to proteins that have the binding properties of the β integrin-based cell adhesion receptor of M. sexta and have an amino acid sequence at least 90% identical to SEQ. ID. No.: 2. The invention is also directed to recombinant materials for the production of this protein. In still other aspects, the invention is directed to methods to identify anti-M. sexta agents by contacting a protein of the invention with a candidate compound and assessing the ability of the candidate compound to bind said protein, whereby a compound that binds the protein is identified as a potential agent for controlling M. sexta infestation. The compound can then be applied to the host plants, or, in some instances, host plants may be modified to produce the compound by transgenic modification.

### BRIEF DESCRIPTION OF THE DRAWINGS

FIG. 1 shows the nucleotide sequence (SEQ. ID. No.: 1) and deduced amino acid sequence (SEQ. ID. No.: 2) of M. sexta β integrin.

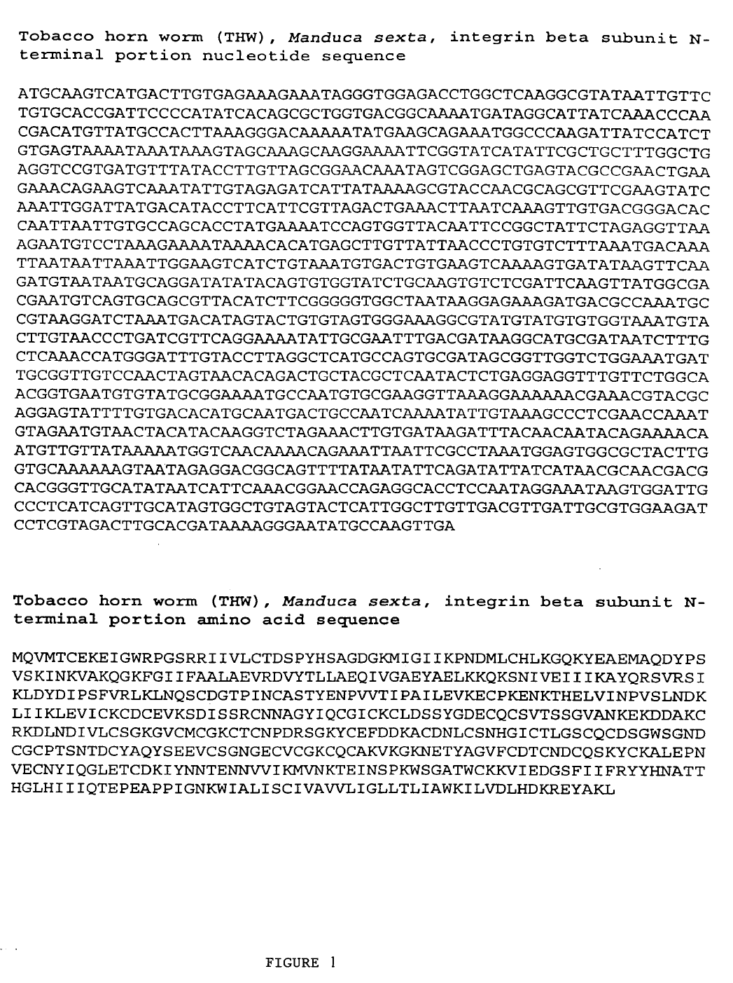

### MODES OF CARRYING OUT THE INVENTION

The integrin family of cell adhesion receptors plays a fundamental role in cell division, differentiation and movement. The receptors are α and β heterodimers wherein the extracellular domains mediate cell-matrix and cell-cell contacts; the cytoplasmic tails associate with the cytoskeleton. Integrins can thus transduce information bi-directionally. The ligand binding region of this receptor is on the β subunit and sequences in the cytoplasmic tails of the β subunits interact with cytoskeletal and signaling components. Green, L. J., et al., Int. J. Biochem. Cell Biol. (1998) 30:179-184.

Integrin β1 associates with α1 or α6 subunits to form a laminin receptor, with α2 to form a collagen receptor, with α4 to interact with VCAM1, with α5 to form a fibronectin receptor and integrin β1 also interacts with α8.

Thus, the ability of a compound to interact with β1 integrin is indicative of its ability to interfere with a variety of integrin-type receptors. Such agents are useful as pesticides in controlling M. sexta and related species.

The present invention provides isolated β1 integrin protein of M. sexta which can be produced, preferably using recombinant techniques, to serve as a screening tool for such pesticide candidates. By “isolated” is meant that the referent is removed from its natural surroundings. It may not necessarily be purified, but is found in an unfamiliar environment. Thus, an “isolated” protein is a protein that may, for example, be displayed on the surface of a heterologous cell; an “isolated” nucleic acid may be included in a recombinant vector that contains heterologous elements.

The nucleotide sequence (SEQ. ID. No.: 1) and deduced amino acid sequence (SEQ. ID. No.: 2) for M. sexta β integrin protein were recovered and determined as follows. M. sexta midgut RNA was extracted and subjected to RT-PCR using primers constructed based on the “gene mining” system described in WO 01/31011 referenced above. The resulting cDNA was subjected to agarose gel electrophoresis and the band of expected size removed. The excised nucleic acids were cloned into a pAT vector and sequenced. BLAST alignment of the sequences identified a clone with similarity to integrin β1 sequences from the signal crayfish, the fruit fly, and the African malaria mosquito.

The retrieved insert was used to clone full-length cDNA from an M. sexta library which resulted in the nucleotide sequence set forth in SEQ. ID. No.: 1. The recovered sequence has 38% identity to the β integrin of the fruit fly (GenBank Accession No. A30889).

The retrieved nucleotide sequence can be inserted into expression vectors and displayed on the surface of host cells. Display on insect host cells is preferred, although screening can also be done using eukaryotic cells in general, such as yeast, mammalian cells, and the like. Expression in prokaryotes can also be effected. A wide variety of expression systems and hosts for recombinantly produced proteins by now is well known in the art.

The displayed β integrin protein may optionally be co-expressed with various α subunits to obtain complete receptors; however, the display of the β1 is sufficient to conduct the screen. Alternatively, the protein of the invention coupled to solid support may be used as a target or a homogeneous assay for interaction of the protein with candidate compound can be used.

The proteins of the invention and corresponding recombinant materials are illustrated by SEQ. ID. No.: 2 and SEQ. ID. No.: 1, respectively. However, a genus of proteins is useful in the screening methods of the invention; this genus is characterized as including proteins which retain the binding specificity of β1 integrin and which have an amino acid sequence at least 90% identical, preferably 95% identical, more preferably 98% identical and still more preferably 99% identical with SEQ. ID. No.: 2. Recombinant materials and methods for the production of these proteins are also included in the invention—thus the invention includes nucleic acids which comprise nucleotide sequences which encode the above proteins, as well as expression systems containing these sequences and host cells containing these nucleic acids.

Nucleic acids comprising at least 15 consecutive nucleotides which hybridize under stringent conditions (i.e., 1×SSC, 0.1×SDS, 60° C. wash conditions) are also useful as probes to obtain β integrin-encoding sequences from related species or from allelic variants of M. sexta β integrin sequences. These probes may also be 90% or more identical to the corresponding region of SEQ. ID. No.: 1 or 95, 98 or 99% identical. Longer probes may also be used with similar restrictions on hybridization and percent identity.

The invention further includes antibodies which are immunoreactive with the proteins of the invention. The term “antibodies” includes both polyclonal and monoclonal antibodies, immunoreactive fragments of these antibodies, such as the F_{ab} fragment, single-chain antibodies, antibodies with variations in regions that do not affect binding specificity, and the like. Methods for producing antibodies both in mammals and recombinantly are well known.

The antibodies of the invention are useful in purifying the invention proteins, as well as in inhibiting the interaction between binding compounds and the integrin receptor. Thus, the antibodies are useful in assays for candidate compounds; for example, the antibodies may be labeled and the ability of the candidate compound to displace label from cells displaying the receptors is used as an index of the ability of the compound to bind the β1 integrin-containing receptor.

The invention thus provides a significant tool for identifying pesticides useful in the control of M. sexta and related species by identifying compounds that react with and bind to an essential component in cell signaling for this species. The pesticides thus developed are applied in standard application procedures to affected plants, typically tobacco plants. In addition, if the pesticide identified is itself a protein, or a compound which is produced by a recognized metabolic pathway, the relevant protein or metabolic path may be produced in situ in the plant to be protected by transforming the cells of the plant with recombinant materials for production of the protein or metabolic pathway.

1. A composition that consists essentially of recombinant host cells which display at their surfaces a protein that exhibits the binding characteristics of M. sexta β integrin which protein has the amino acid sequence of SEQ. ID. No.: 2, and wherein said protein is produced from an expression system for the production of said protein heterologous to said recombinant host cells.

2. The composition of claim 1 wherein the cells are insect cells.

3. The composition of claim 1 wherein the cells are mammalian or yeast cells.

4. A method to identify a compound as a candidate pesticide effective against M. sexta, which method comprises contacting a candidate compound with the composition of claim 1;
assessing the binding of said compound to said cells;
whereby a compound which binds said cells is identified as a candidate pesticide against M. sexta.


SEQUENCE LISTING (2 sequences)

SEQ ID NO: 1 - DNA, 1513 bp, Manduca sexta
atgcaagtcatgacttgtgagaaagaaatagggtggagacctggctcaaggcgtataattgttctgtgcaccgattccccatatcacagcgctggtgacggcaaaatgataggcattatcaaacccaacgacatgttatgccacttaaagggacaaaaatatgaagcagaaatggcccaagattatccatctgtgagtaaaataaataaagtagcaaagcaaggaaaattcggtatcatattcgctgctttggctgaggtccgtgatgtttataccttgttagcggaacaaatagtcggagctgagtacgccgaactgaagaaacagaagtcaaatattgtagagatcattataaaagcgtaccaacgcagcgttcgaagtatcaaattggattatgacataccttcattcgttagactgaaacttaatcaaagttgtgacgggacaccaattaattgtgccagcacctatgaaaatccagtggttacaattccggctattctagaggttaaagaatgtcctaaagaaaataaaacacatgagcttgttattaaccctgtgtctttaaatgacaaattaataattaaattggaagtcatctgtaaatgtgactgtgaagtcaaaagtgatataagttcaagatgtaataatgcaggatatatacagtgtggtatctgcaagtgtctcgattcaagttatggcgacgaatgtcagtgcagcgttacatcttcgggggtggctaataaggagaaagatgacgccaaatgccgtaaggatctaaatgacatagtactgtgtagtgggaaaggcgtatgtatgtgtggtaaatgtacttgtaaccctgatcgttcaggaaaatattgcgaatttgacgataaggcatgcgataatctttgctcaaaccatgggatttgtaccttaggctcatgccagtgcgatagcggttggtctggaaatgattgcggttgtccaactagtaacacagactgctacgctcaatactctgaggaggtttgttctggcaacggtgaatgtgtatgcggaaaatgccaatgtgcgaaggttaaaggaaaaaacgaaacgtacgcaggagtattttgtgacacatgcaatgactgccaatcaaaatattgtaaagccctcgaaccaaatgtagaatgtaactacatacaaggtctagaaacttgtgataagatttacaacaatacagaaaacaatgttgttataaaaatggtcaacaaaacagaaattaattcgcctaaatggagtggcgctacttggtgcaaaaaagtaatagaggacggcagttttataatattcagatattatcataacgcaacgacgcacgggttgcatataatcattcaaacggaaccagaggcacctccaataggaaataagtggattgccctcatcagttgcatagtggctgtagtactcattggcttgttgacgttgattgcgtggaagatcctcgtagacttgcacgataaaagggaatatgccaagttga

SEQ ID NO: 2 - protein, 504 aa, Manduca sexta
MQVMTCEKEIGWRPGSRRIIVLCTDSPYHSAGDGKMIGIIKPNDMLCHLKGQKYEAEMAQDYPSVSKINKVAKQGKFGIIFAALAEVRDVYTLLAEQIVGAEYAELKKQKSNIVEIIIKAYQRSVRSIKLDYDIPSFVRLKLNQSCDGTPINCASTYENPVVTIPAILEVKECPKENKTHELVINPVSLNDKLIIKLEVICKCDCEVKSDISSRCNNAGYIQCGICKCLDSSYGDECQCSVTSSGVANKEKDDAKCRKDLNDIVLCSGKGVCMCGKCTCNPDRSGKYCEFDDKACDNLCSNHGICTLGSCQCDSGWSGNDCGCPTSNTDCYAQYSEEVCSGNGECVCGKCQCAKVKGKNETYAGVFCDTCNDCQSKYCKALEPNVECNYIQGLETCDKIYNNTENNVVIKMVNKTEINSPKWSGATWCKKVIEDGSFIIFRYYHNATTHGLHIIIQTEPEAPPIGNKWIALISCIVAVVLIGLLTLIAWKILVDLHDKREYAKL